(19) United States
(12) Patent Application Publication
(10) Pub. No.: US 2019/0361032 A1
(43) Pub. Date: Nov. 28, 2019

(54) AUTOMATED DRUG ASSAYS

(71) Applicant: Virginia Commonwealth University, Richmond, VA (US)

(72) Inventors: Sudha Jayaraman, Richmond, VA (US); Dayanjan S. Wijesinghe, Richmond, VA (US)

(21) Appl. No.: 16/361,640

(22) Filed: Mar. 22, 2019

Related U.S. Application Data
(60) Provisional application No. 62/676,846, filed on May 25, 2018.

(51) Int. Cl. G01N 33/68; G01N 30/72; G01N 30/88; H01J 49/04; G16H 20/10

(52) U.S. Cl. CPC G01N 33/6848; G01N 30/7233; G01N 30/88; G01N 2030/8822; G16H 20/10; H01J 49/0413; H01J 49/0431

(57) ABSTRACT

Disclosed are various embodiments for automated drug assays. A command is sent to the mass spectrometer to analyze a blood sample to generate a mass spectrum. The mass spectrum is then received from the mass spectrometer. The mass spectrum is analyzed to determine an identity of a pharmaceutical compound present in the blood sample. Finally, a report for the blood sample is generated, the report containing the identity of the pharmaceutical compound present in the blood sample.

### CROSS-REFERENCE TO RELATED APPLICATIONS

This application claims priority to, and the benefit or, U.S. Provisional Patent Application No. 62/676,846, entitled “AUTOMATED DRUG ASSAYS” and filed on May 25, 2018, which is incorporated by reference as if set forth herein in its entirety.

### GOVERNMENT SUPPORT

This invention was made with government support under grant numbers HD087198 and P60MD002256 awarded by the National Institutes of Health. The government has certain rights in the invention.

### BACKGROUND

Doctors are expected to know what drugs, medications, or other pharmaceutical or pharmacological compounds are in a patient's body. This information can be critical to the patient's ultimate outcome as certain medications, when combined with other medications, can produce lethal effects. For example, the combination of alcohol and acetaminophen in an individual can cause acute liver-failure. Therefore, a doctor may wish to know how much alcohol, if any, is in a patient's blood before prescribing a pain medication that contains acetaminophen, such as VICODIN® (a prescription combination of hydrocodone and acetaminophen). Likewise, the combinations of some non-steroid anti-inflammatory drugs (NSAIDs), such as aspirin or ibuprofen, blood-thinners (e.g., dabigatran), and clot-busting medications (e.g., tissue plasminogen activators) can result in internal hemorrhaging. So a doctor treating a stroke victim may need to know whether the patient has recently taken an NSAID or blood-thinner prior to prescribing a clot-buster to treat the stroke.

Historically, physicians have relied upon the patient, the patient's family, or emergency medical technicians (EMT) or the patient's primary physician or pharmacy to provide the doctor with a list of medications or drugs that the patient has recently taken. However, these approaches are often inaccurate. A patient may forget to mention a medication that he or she has taken, how long ago it was taken, or the dose of the medication. A patient may also not want to admit to using a medication they were not prescribed (e.g., a spouse's medication), even though doctors need to know about all of a patient's medications that he or she has recently used in order to avoid prescribing a medication that could interact in an adverse manner with what a patient has recently taken. Often, patients may also be incapacitated or otherwise unable to communicate. In these situations, EMTs or family members may inform the emergency room physician of any medications that they are aware of the patient taking. But these individuals are even less likely to have a complete knowledge of the patient's medication history. For example, they may provide the doctor with a collection of prescription drugs collected from the patient (e.g., all the drugs in the patient's medicine cabinet), but this only provides a list of medications that the patient may be taking, rather than a list of the medications that the patient has actually taken recently.

Moreover, standard blood tests for medications often take weeks to perform. However, decisions in an emergency setting often need to be made within seconds or minutes. As a result, an emergency room physician must often make decisions about what medication to prescribe based on incomplete information provided by the patient, the patient's family, or EMTs.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing depicting one of several embodiments of the present disclosure.

FIG. 2A is a drawing depicting one or more components used with various embodiments of the present disclosure.

FIG. 2B is a drawing depicting one or more components used with various embodiments of the present disclosure.

FIG. 2C is a drawing depicting one or more components used with various embodiments of the present disclosure.

FIG. 3 is a schematic block diagram of a computing device depicted in FIG. 1 according to various embodiments of the present disclosure.

FIG. 4 is a flowchart illustrating one example of functionality implemented as portions of an application executed by the computing device depicted in FIG. 3.

FIG. 5 is a flowchart illustrating one example of functionality implemented as portions of an application executed by the computing device depicted in FIG. 3.

### DETAILED DESCRIPTION

Disclosed are various embodiments for automated drug assays. Blood drawn from a patient is inserted into a consumable container, which includes one or more radio frequency identification (RFID) sensors. The consumable container is then inserted into a chromatograph. The chromatograph verifies the contents of the consumable container and one or more properties of the consumable container. For example, the chromatograph could use an RFID reader to obtain values from the one or more RFID sensors of the consumable container. The chromatograph may also perform a self-test to verify that it is able to process the consumable container prior to beginning the drug assay process. Once the chromatograph verifies the contents of the consumable container, it then separates the blood sample into individual components, which are fed into a mass spectrometer. The mass spectrometer then analyzes the blood sample. The result is then compared to a catalog of pharmaceutical or pharmacological compounds to identify individual compounds in the blood sample. A report is then generated listing all of the pharmacological or pharmaceutical compounds present in the blood sample. The report can then be sent to another computing device (e.g., a hospital's patient management system or electronic medical records system, a physician's smartphone or tablet, or other computing device). In the following discussion, a general description of the system and its components is provided, followed by a discussion of the operation of the same.

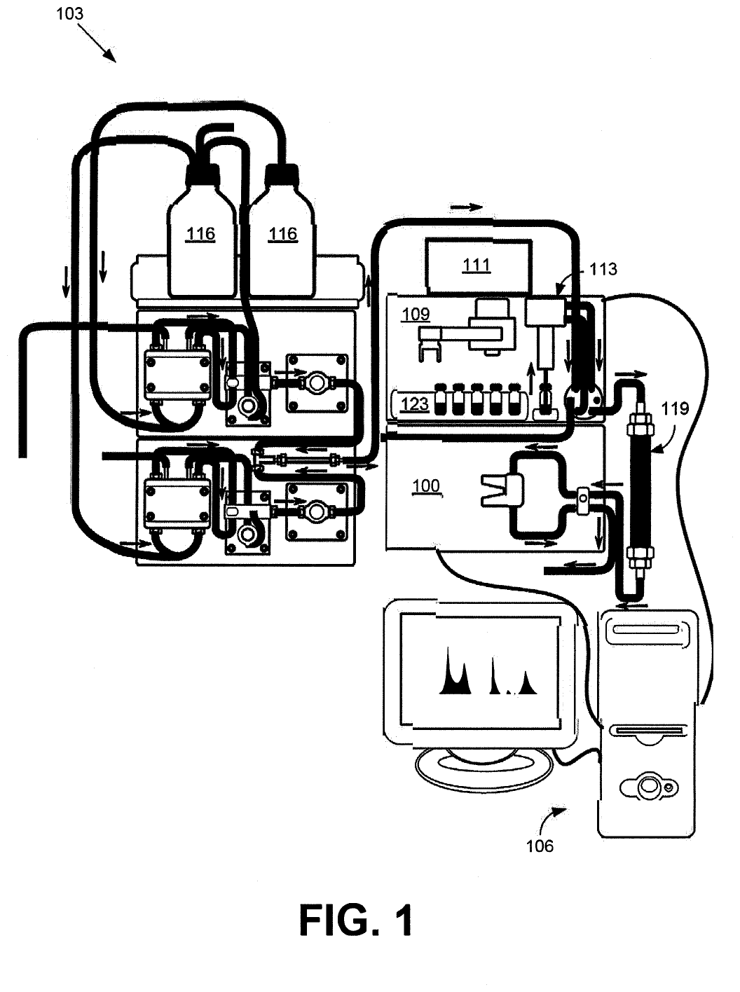

FIG. 1 depicts an example implementation according to the principles of the present disclosure. A mass spectrometer 100 is connected to a high-performance liquid-chromatograph 103. Both a high-performance liquid-chromatograph 103 and the mass spectrometer 100 are in data communication with a computing device 106. Although a high-performance liquid-chromatograph 103 is depicted, other chromatographs may be used to separate a blood sample into individual components for analysis by the mass spectrometer 100. Examples of alternative chromatographs that could be used include gas chromatographs and liquid chromatographs. In some embodiments, other separation devices can be used in place of the high-performance liquid-chromatograph 103, such as an ion-mobility spectrometer or an apparatus that uses capillary electrophoresis.

The high-performance liquid-chromatograph 103 can include a number of components. For example, the high-performance liquid-chromatograph 103 can include an auto-sampler 109. In some embodiments, the high-performance liquid-chromatograph 103 can also include a radio frequency identifier (RFID) reader 111.

The auto-sampler 109 can automatically load one or more blood samples for extraction into a guard column 113 where the blood sample is combined with one or more solvents obtained from respective solvent containers 116. For example, the auto-sampler 109 can include a robotic arm or other apparatus for automatically loading blood samples for further analysis. After combining with the solvent(s) obtained from the solvent container(s) 116, the loaded blood sample can then flow from the guard column 113 through an analytical column 119 before entering the mass spectrometer 100. In some embodiments, the auto-sampler 109 can also include a sensor 123 that determines when a container containing a blood sample has been placed within the auto-sampler 109. As an example, the sensor 123 could be a pressure or weight sensor that detects when a container has been placed on top of a surface in the auto-sampler 109.

The RFID reader 111 can be used by the high-performance liquid-chromatograph 103 to perform self-tests prior to analyzing a blood sample. For example, the RFID reader 111 can be used to read RFID tags or sensors affixed to various components to confirm that all of the components are able to be used to analyze the blood sample.

The computing device 106 can be used to perform various operations. For example, the computing device 106 can be used to control the operation of the high-performance liquid-chromatograph 103 and the mass spectrometer 100. As another example, the computing device 106 can be used to receive and process data from the high-performance liquid-chromatograph 103 or mass spectrometer 100. The computing device 106 can also be used to generate various reports from the received or processed data and share the reports or data with various other computing devices or applications.

The computing device 106 is representative of one or more computing devices 106 that may be in data communication with the high-performance liquid-chromatograph 103 or mass spectrometer 100. The computing device 106 may include, for example, a processor-based system be embodied in the form of a personal computer (e.g., a desktop computer, a laptop computer, or similar device), a mobile computing device (e.g., personal digital assistants, cellular telephones, smartphones, web pads, tablet computer systems, and similar devices), or other devices with like capability. The computing device 106 may include one or more displays, such as liquid crystal displays (LCDs), gas plasma-based flat panel displays, organic light emitting diode (OLED) displays, electrophoretic ink (“E-ink”) displays, projectors, or other types of display devices. In some instances, the display may be a component of the computing device 106 or may be connected to the computing device 106 through a wired or wireless connection.

The computing device 106 may be configured to execute various applications computing device 106. These applications may be executed to send commands to the high-performance liquid-chromatograph 103 or the mass spectrometer 100. These applications may also be executed to receive and process data from the high-performance liquid-chromatograph 103 or the mass spectrometer 100. In addition, these applications may be executed to generate a report from the received and processed data. These applications may further be executed to cause the computing device 106 to share or otherwise send the generated reports with another computing device or system. To this end, these applications may include, for example, a browser, a dedicated application, or other executable. Moreover, these applications may cause a user interface to be rendered on the display of the computing device 100 to allow a user to submit user input in the form of commands, queries or requests. For example, the user interface could allow a user to start or halt operation of the high-performance liquid-chromatograph 103 or the mass spectrometer 100. Moreover, the user interface could allow the user to select a report to generate or specify which computing devices, systems, or applications to share the generated report with. To this end, the user interface may include a web page, an application screen, other user mechanism for obtaining user input.

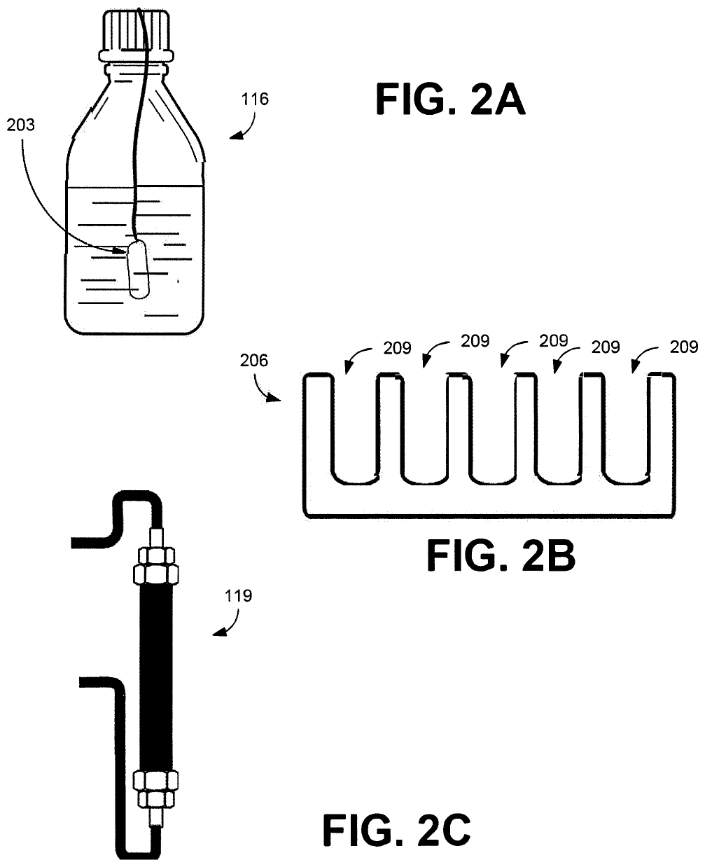

FIG. 2A depicts an example of a solvent container 116 according to various embodiments of the present disclosure. The solvent container 116 can include an RFID sensor 203. In some embodiments, the RFID sensor 203 may be a depth sensor that can transmit a measure of the amount of solvent in the solvent container 116 when the RFID sensor 203 is activated by an RFID reader 111 (FIG. 1). In other embodiments, the RFID sensor 203 may, when activated by the RFID reader 111, determine whether the solvent container 116 has been opened (e.g., by detecting that a wire affixed to a seal or opening of the solvent container 116 has been broken).

FIG. 2B depicts an example of a test cartridge 206 according to various embodiments of the present disclosure. The test cartridge 206 can include one or more compartments 209. In some embodiments, the compartments 209 may be glass-lined or contain glass inserts. One or more samples of a known pharmaceutical or pharmacological compound may be stored in individual compartments of the test cartridge 206. As further described herein, these samples of known pharmaceutical or pharmacological compounds can be used to calibrate the mass spectrometer 100 and the high-performance liquid-chromatograph 103 in order to verify their accuracy prior to analysis of a blood sample.

FIG. 2C depicts an example of an analytical column 119 according to various embodiments of the present disclosure.

One or more of the solvent container 116, test cartridge 206, and analytical column 119 depicted in FIG. 2A, FIG. 2B, and FIG. 2C may be prepackaged together as disposable or consumable test kit. In some embodiments, the solvent container 116, test cartridge 206, and the analytical column 119 may be combined with a container for a blood sample to form the disposable or consumable test kit.

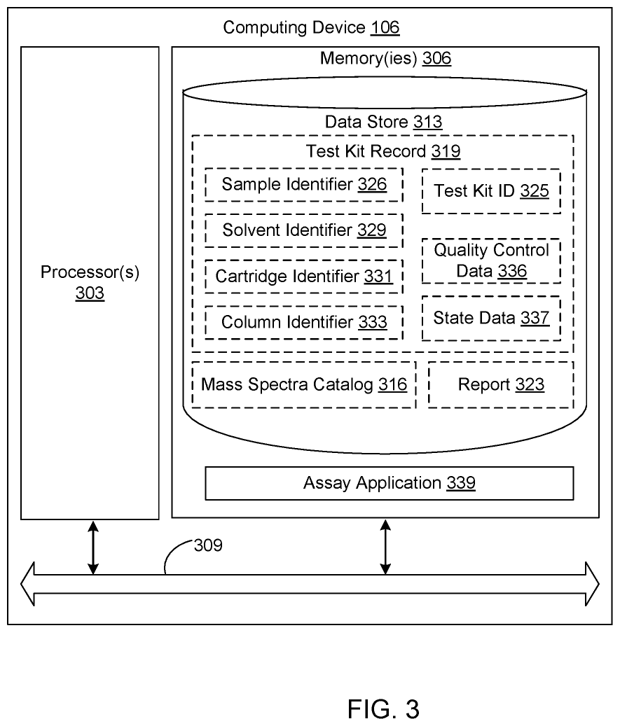

FIG. 3 depicts a schematic block diagram of the computing device 106 according to various embodiments of the present disclosure. The computing device 106 includes at least one processor circuit, for example, having a processor 303 and a memory 306, both of which are coupled to a local interface 309. The local interface 309 may include, for example, a data bus with an accompanying address/control bus or other bus structure as can be appreciated.

Stored in the memory 306 are both data and several components that are executable by the processor 303. In particular, stored in the memory 306 and executable by the processor 303 are list of main applications, and potentially other applications. Also stored in the memory 306 may be a data store 313 and other data. In addition, an operating system may be stored in the memory 306 and executable by the processor 303.

The data store 313 may be representative of a plurality of data stores 313, which can include relational databases, object-oriented databases, hierarchical databases, hash tables or similar key-value data stores, as well as other data storage applications or data structures. The data stored in the data store 313 is associated with the operation of the various applications or functional entities described below. This data can include a mass spectra catalog 316, one or more test kit records 319, and one or more reports 323, and potentially other data.

The mass spectra catalog 316 represents a catalog of mass spectrum readings of previously identified pharmaceutical or pharmacological compounds. Each entry in the catalog can include the name of a known pharmaceutical or pharmacological compound and a corresponding mass spectrum reading for the pharmaceutical or pharmacological compound.

The test kit record 319 represents information about a test kit and its components, as used in various embodiments of the present disclosure. Each test kit record 319 can include a test kit identifier 325 that uniquely identifies a test kit. Each test kit record 319 can also include a sample identifier 326 for a blood sample container associated with the test kit, a solvent identifier 329 for each solvent container 116 (FIG. 1 and FIG. 2A) associated with the test kit, a cartridge identifier 331 for each test cartridge 206 (FIG. 2B) associated with a test kit, and a column identifier 333 for each analytical column 119 (FIG. 1 and FIG. 2C) associated with the test kit. The test kit record can also include quality control data 336 for the test kit and state data 337 for the test kit. The quality control data 336 includes mass spectra results for known compound included in the test cartridge 206, the minimum amount of a solvent necessary to perform a test of a blood sample using the test kit, a maximum number of measurements that can be made using the analytical column 119 associated with the test kit, and similar data. State data 337 includes information about the current state of the test kit. This information can include historic data such as whether a solvent container 116 has been previously opened or the amount of solvent used in an opened solvent container 116, the number of measurements previously made with the analytical column 119, the maximum number of measurements that can be made with the analytical column 119, the expiration date for the test kit, and potentially other data.

The report 323 represents a result of a test of a blood sample using the mass spectrometer 100 and the high-performance liquid-chromatograph 103. The report 323 may be generated by the assay application 339, as later described. The report 323 can include a list of pharmaceutical or pharmacological compounds determined to be present in the blood sample, and potentially the amount of the pharmaceutical or pharmacological compounds present. The report 323 may contain additional information according to various embodiments of the present disclosure.

As previously discussed, various applications or other components may be executed by the processor 303 of the computing device 106 according to various embodiments. The applications executed by the processor 303 can include the assay application 339. It is understood that there may be other applications that are stored in the memory 306 and are executable by the processor 303. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

The assay application 339 is executed to perform a number of operations related to the various embodiments of the present disclosure. For example, the assay application 339 can be executed to control the operation of the mass spectrometer 100 and the high-performance liquid-chromatograph 103. For instance, the assay application 339 may be used to cause the mass spectrometer 100 and the high-performance liquid-chromatograph 103 to process a blood sample. The assay application 339 may also be used to initiate a self-test of the mass spectrometer 100 and the high-performance liquid-chromatograph 103 prior to testing a blood sample. The assay application 339 can also be used to receive and process the data generated by the mass spectrometer 100 and the high-performance liquid-chromatograph 103 as a result of testing the blood sample. This can include identifying pharmaceutical or pharmacological compounds in the mass spectra catalog 316 that match the results of testing the blood sample and generating a resulting report 313 that includes the identified pharmaceutical or pharmacological compounds. The assay application 339 can also be used to share reports 323. For instance, the assay application 339 can be used to share or otherwise send a report 323 to another computing device or application.

The term “executable” means a program file that is in a form that can ultimately be run by the processor 303. Examples of executable programs include a compiled program that can be translated into machine code in a format that can be loaded into a random access portion of the memory 306 and run by the processor 303, source code that may be expressed in proper format such as object code that is capable of being loaded into a random access portion of the memory 306 and executed by the processor 303, or source code that may be interpreted by another executable program to generate instructions in a random access portion of the memory 306 to be executed by the processor 303, etc. An executable program may be stored in any portion or component of the memory 306 including, for example, random access memory (RAM), read-only memory (ROM), hard drive, solid-state drive, Universal Serial Bus (USB) flash drive, memory card, optical disc such as compact disc (CD) or digital versatile disc (DVD), floppy disk, magnetic tape, or other memory components.

The memory 306 is defined herein as including both volatile and nonvolatile memory and data storage components. Volatile components are those that do not retain data values upon loss of power. Nonvolatile components are those that retain data upon a loss of power. Thus, the memory 306 may include, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, or other memory components, or a combination of any two or more of these memory components. In addition, the RAM may include, for example, static random access memory (SRAM), dynamic random access memory (DRAM), or magnetic random access memory (MRAM) and other such devices. The ROM may include, for example, a programmable read-only memory (PROM), an erasable programmable read-only memory (EPROM), an electrically erasable programmable read-only memory (EEPROM), or other like memory device.

Also, the processor 303 may represent multiple processors 303 or multiple processor cores and the memory 306 may represent multiple memories 306 that operate in parallel processing circuits, respectively. In such a case, the local interface 309 may be an appropriate network that facilitates communication between any two of the multiple processors 303, between any processor 303 and any of the memories 306, or between any two of the memories 306. The local interface 309 may include additional systems designed to coordinate this communication, including, for example, performing load balancing. The processor 303 may be of electrical or of some other available construction.

Although the assay application 339, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Next, a general description of the operation of the various components of the present disclosure is provided. More detailed descriptions of the operation of specific components is provided later in this application.

To begin, a sample of blood is collected from a patient. In some embodiments, the blood sample may be preprocessed prior to testing to remove various biological components. For example the blood sample may be treated with an anticoagulant and spun in a centrifuge to separate blood cells from the blood plasma. The blood plasma could then be test by the mass spectrometer 100 and the high-performance liquid-chromatograph 103. In some of these embodiments, the blood plasma could be further treated to remove proteins in suspension. However, in some embodiments, the blood sample may be tested using the mass spectrometer 100 and the high-performance liquid-chromatograph 103 without any preprocessing.

Next, a user can use a test kit to prepare the blood sample for testing. For example, the user can attach solvent containers 116 included in a test kit to the high-performance liquid-chromatograph 103. The user can then connect the high-performance liquid-chromatograph 103 to the mass spectrometer 100 using the analytical column 119 included in the test kit. Finally, the user can insert a container (e.g. a vial, beaker, or similar container) containing the blood sample and a test cartridge 206 into the auto-sampler 109 (FIG. 1).

In some embodiments, the sensor 123 detects the insertion of the container and the test cartridge 206 and automatically initiates a self-test prior to processing the blood sample. In other embodiments, the user may initiate the self-test through a user interface rendered by the assay application 339 on a display of the computing device 106.

During the self-test, the mass spectrometer 100 and the high-performance liquid-chromatograph 103 verify their accuracy and the quality of the test kit through several operations. First, the RFID reader 111 (FIG. 1) obtains the identifiers of all of the test kit components and provides them to the assay application 339. The assay application 339 then confirms, based on the identifiers received from the RFID reader 111, that the test kit components are all from the same test kit. Then, the RFID reader 111 can read the values of the RFID sensors 203 (FIG. 2A) of the solvent containers 116 to determine whether the solvent containers 116 have been previously opened and the amount of solvent in each solvent container 116. This information is further reported back to the assay application 339, which confirms that there is sufficient solvent to test the blood sample. The assay application 339 may also consult the test kit record 319 for the respective test kit to determine whether the test kit has expired.

The auto-sampler 109 then loads the test cartridge 206 for the mass spectrometer 100 and the high-performance liquid-chromatograph 103 to process. The resulting data is then provided to the assay application 339, which compares the measured results to the quality control data 336 for the respective test kit. If the resulting data falls within the ranges defined by the quality control data 336, then the assay application 339 can determine that the mass spectrometer 100 and the high-performance liquid-chromatograph 103 are operating correctly.

The assay application 339 then sends a command to the mass spectrometer 100 and the high-performance liquid-chromatograph 103 to process the blood sample. In response, the auto-sampler loads the container containing the blood sample for the mass spectrometer 100 and the high-performance liquid-chromatograph 103 to process. Mass spectrum data is collected and provided to the assay application 339, which compares the mass spectrum data to individual entries in a mass spectra catalog 316. For each entry in the mass spectra catalog 316 that matches the collected mass spectrum data, the assay application records in a report 323 that the corresponding pharmaceutical or pharmacological compound is present in the blood sample. The report is then saved to the data store 313.

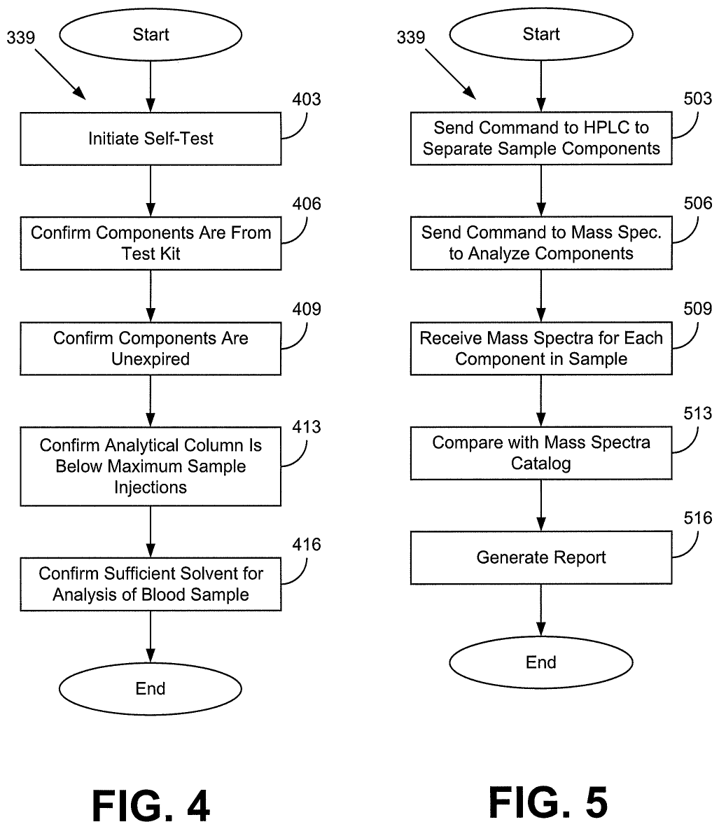

Referring next to FIG. 4, shown is a flowchart that provides one example of the operation of a portion of the assay application 339 according to various embodiments. It is understood that the flowchart of FIG. 4 provides merely an example of the many different types of functional arrangements that may be employed to implement the operation of the portion of the assay application 339 as described herein. As an alternative, the flowchart of FIG. 4 may be viewed as depicting an example of elements of a method implemented in the computing device 106 (FIG. 1 and FIG. 3) according to one or more embodiments.

Beginning with box 403, the assay application 339 initiates a self-test. The self-test may be initiated in response to receiving a notification from the auto-sampler 109 that a blood sample container or a test cartridge 206 have been placed in the auto-sampler 109. For example, the weight of a blood sample container or a test cartridge 206 may activate a sensor 123, such as a pressure sensor. To initiate the self-test, the assay application 339 can send a command to the high-performance liquid-chromatograph 103 to use the RFID reader 111 (FIG. 1) to read the individual RFID tags affixed to the individual components of the test kit. Each RFID tag may respond with a test kit identifier 325 of the test kit that the component is from, as well as a respective sample identifier 326, solvent identifier 329, cartridge identifier 331, and column identifier 333.

Moving to box 406, the assay application 339 receives test kit identifier(s) 325, the sample identifier 326, solvent identifier 329, cartridge identifier 331, and column identifier 333 from the RFID reader 111. The assay application 339 can then verify that the blood sample container, the solvent container(s) 116, the test cartridge 206, and the analytical column 119 are from the same test kit. For example, the assay application 339 may verify that each component reported the same test kit identifier 325. If more than one test kit identifier 325 is received, then the assay application 339 can determine that not all of the components are from the same test kit and the self-test will fail. As another example, the assay application 339 can query the respective test kit record 319 for the test kit identifier 325 and determine whether the received sample identifier 326, solvent identifier 329, cartridge identifier 331, and column identifier 333 match the corresponding values in the test kit record 319. If there is a mismatch, then the assay application 339 can determine that not all of the components are from the same test kit and the self-test will fail.

Proceeding to box 409, the assay application 339 confirms that the components of the test kit are not expired. For example, the assay application 339 can compare the current date to an expiration date specified in the state data 337 linked to the test kit record 319 identified by the received test kit identifier 325. If the current date is after the expiration date, then the assay application 339 can determine that the test kit is expired and the self-test will fail.

Next at box 413, the assay application 339 confirms that the number of injections for which the analytical column 119 has been used is below some maximum threshold. For example, the assay application 339 can query the state data 337 to determine if the analytical column 119 has been used before, the number of injections for which the analytical column 119 has been used, and compare it to a maximum value specified by the quality control data 335. If the maximum value is exceeded, the self-test will fail.

Finally, a box 416 the assay application 339 confirms that there is sufficient solvent in the solvent containers 116 to analyze the blood sample. For example, the assay application 339 may send a command to the RFID reader 111 to obtain the values reported by the RFID sensor(s) 203 (FIG. 2A) of the solvent containers 116. The assay application 339 can then analyze the values reported by the RFID sensor(s) 203. For instance, if the value reported by the RFID sensor(s) 203 indicate that the depth of the solvent in the solvent containers 116 are below a minimum level, the assay application 339 can determine that there is insufficient solvent for analyzing the blood sample and the self-test will fail.

In the event that the assay application 339 completes the process defined by boxes 403-416 without the self-test failing, then the assay application 339 can deem that the self-test passed.

Referring next to FIG. 5, shown is a flowchart that provides one example of the operation of a portion of the assay application 339 according to various embodiments. It is understood that the flowchart of FIG. 5 provides merely an example of the many different types of functional arrangements that may be employed to implement the operation of the portion of the assay application 339 as described herein. As an alternative, the flowchart of FIG. 5 may be viewed as depicting an example of elements of a method implemented in the computing device 106 (FIG. 1) according to one or more embodiments.

Beginning with box 503, the assay application 339 sends a command to the high-performance liquid-chromatograph 103 to begin analysis of the blood sample by separating the blood sample into its constituent component compounds and molecules. When the high-performance liquid-chromatograph 103 receives the command, the auto-sampler 109 loads the container containing the blood sample into the guard column 113. The blood sample is then extracted from the container and mixed with one or more solvents before entering the analytical column 119.

Next at box 506, the assay application 339 sends a command to the mass spectrometer 100 to begin analyzing the blood sample. As molecules enter the mass spectrometer 100 from the analytical column 119, the mass spectrometer 100 ionizes the molecules and measures the mass spectrum of each molecule. The mass spectrometer 100 reports the measured mass spectrum of each molecule back to the assay application 339. Accordingly, at box 509, the assay application 339 receives and stores the received mass spectrum data for each molecule in the blood sample.

Proceeding to box 513, the assay application 339 compares the mass spectrum data for each molecule to the entries in the mass spectrum catalog 316. If an entry in the mass spectrum catalog 316 matches the mass spectrum data for a received molecule, the identity of the molecule listed in the entry in the mass spectrum catalog 316 is recorded.

Next at box 516, the assay application 339 generates a report 323. The report includes the identity of each matching molecule identified in the mass spectra catalog 316 at box 519. The report is then saved in the data store 313 (FIG. 3) for future use.

The flowcharts of FIGS. 4 and 5 show the functionality and operation of an implementation of portions of the assay application 339. If embodied in software, each block may represent a module, segment, or portion of code that includes program instructions to implement the specified logical function(s). The program instructions may be embodied in the form of source code that includes human-readable statements written in a programming language or machine code that includes numerical instructions recognizable by a suitable execution system such as a processor 303 in a computer system or other system. The machine code may be converted from the source code through various processes. For example, the machine code may be generated from the source code with a compiler prior to execution of the corresponding application. As another example, the machine code may be generated from the source code concurrently with execution with an interpreter. Other approaches can also be used. If embodied in hardware, each block may represent a circuit or a number of interconnected circuits to implement the specified logical function or functions.

Although the flowcharts of FIGS. 4 and 5 show a specific order of execution, it is understood that the order of execution may differ from that which is depicted. For example, the order of execution of two or more blocks may be scrambled relative to the order shown. Also, two or more blocks shown in succession in FIGS. 4 and 5 may be executed concurrently or with partial concurrence. Further, in some embodiments, one or more of the blocks shown in FIGS. 4 and 5 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the assay application 339, that includes software or code can be embodied in any non-transitory computer-readable medium for use by or in connection with an instruction execution system such as, for example, a processor 303 in a computer system or other system. In this sense, the logic may include, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can include any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including list of main applications, may be implemented and structured in a variety of ways. For example, one or more applications described may be implemented as modules or components of a single application. Further, one or more applications described herein may be executed in shared or separate computing devices or a combination thereof.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiments without departing substantially from the spirit and principles of the disclosure. All such modifications and variations are intended to be included herein within the scope of this disclosure and protected by the following claims.

Therefore, the following is claimed:

1. A system, comprising:
a mass spectrometer;
a high-performance liquid-chromatograph coupled to the mass spectrometer;
a computing device in data communication with the mass spectrometer and the high-performance liquid-chromatograph, the computing device comprising a processor and a memory; and
machine readable instructions stored in the memory that, when executed by the processor, cause the computing device to at least:
send a first command to the high-performance liquid-chromatograph to separate individual components of a blood sample;
send a second command to the mass spectrometer to analyze the individual components of the blood sample to generate a mass spectrum for each of the individual components of the blood sample;
receive the mass spectrum for each of the individual components from the mass spectrometer;
analyze the mass spectrum for each of the individual components to determine an identity of a pharmaceutical compound present in the blood sample; and
generate a report for the blood sample, the report containing the identity of the pharmaceutical compound present in the blood sample.

2. The system of claim 1, wherein the machine readable instructions that cause the computing device to analyze the mass spectrum for each of the individual components to determine the identity of the pharmaceutical compound present in the blood sample further cause the computing device to at least:
compare the mass spectrum to a catalog of mass spectra, the catalog of mass spectra comprising a plurality of entries, each entry representing a mass spectrum of an identified pharmaceutical compound; and
determine that one of the plurality of entries in the catalog of mass spectra matches the mass spectrum received from the mass spectrometer.

3. The system of claim 1, wherein the high-performance liquid-chromatograph comprise an auto-sampler and the machine readable instructions, when executed by the processor, further cause the computing device to:
receive a first notification from an auto-sampler of the high-performance liquid-chromatograph that the blood sample has been inserted into the auto-sampler;
in response to receipt of the notification, send a third command to the high-performance liquid-chromatograph and the mass spectrometer to initiate a self-test; and
determine that the high-performance liquid-chromatograph and the mass spectrometer passed the self-test.

4. The system of claim 3, wherein the machine-readable instructions that cause the computing device to send the first command to the high-performance liquid-chromatograph to separate the individual components of the blood sample in response to a determination that the high-performance liquid-chromatograph and the mass spectrometer passed the self-test.

5. The system of claim 3, wherein the high-performance liquid-chromatograph comprises a radio frequency identification (RFID) reader and the high-performance liquid-chromatograph, in response to receipt of the third command to initiate the self-test, is configured to at least:
read an RFID sensor affixed to a solvent container to determine a level of solvent in the solvent container; and
determine that the level of solvent in the solvent container is sufficient to separate the individual components of the blood sample.

6. The system of claim 1, wherein the machine readable instructions, when executed by the processor, further cause the computing device to at least send the report to a remote computing device selected through a user interface rendered by the machine readable instructions on a display of the computing device.

7. The system of claim 1, wherein the machine readable instructions, when executed by the processor, further cause the computing device to at least render a user interface on a display connected to the computing device, the user interface providing an option to select the pharmaceutical compound to identify in the blood sample.

8. A non-transitory computer-readable medium comprising machine readable instructions that, when executed by a processor of a computing device, cause the computing device to at least:
send a first command to a high-performance liquid-chromatograph to separate individual components of a blood sample;
send a second command to a mass spectrometer to analyze the individual components of the blood sample to generate a mass spectrum for each of the individual components of the blood sample;
receive the mass spectrum for each of the individual components from the mass spectrometer;
analyze the mass spectrum for each of the individual components to determine an identity of a pharmaceutical compound present in the blood sample; and
generate a report for the blood sample, the report containing the identity of the pharmaceutical compound present in the blood sample.

9. The non-transitory computer-readable medium of claim 8, wherein the machine readable instructions that cause the computing device to analyze the mass spectrum for each of the individual components to determine the identity of the pharmaceutical compound present in the blood sample further cause the computing device to at least:
compare the mass spectrum to a catalog of mass spectra, the catalog of mass spectra comprising a plurality of entries, each entry representing a mass spectrum of an identified pharmaceutical compound; and
determine that one of the plurality of entries in the catalog of mass spectra matches the mass spectrum received from the mass spectrometer.

10. The non-transitory computer-readable medium of claim 8, wherein the machine readable instructions, when executed by the processor, further cause the computing device to:
receive a first notification from an auto-sampler of the high-performance liquid-chromatograph that the blood sample has been inserted into the auto-sampler;
in response to receipt of the notification, send a third command to the high-performance liquid-chromatograph and the mass spectrometer to initiate a self-test; and
determine that the high-performance liquid-chromatograph and the mass spectrometer passed the self-test.

11. The non-transitory computer-readable medium of claim 10, wherein the machine-readable instructions that cause the computing device to send the first command to the high-performance liquid-chromatograph to separate the individual components of the blood sample in response to a determination that the high-performance liquid-chromatograph and the mass spectrometer passed the self-test.

12. The non-transitory computer-readable medium of claim 8, wherein the machine readable instructions, when executed by the processor, further cause the computing device to at least send the report to a remote computing device selected through a user interface rendered by the machine readable instructions on a display of the computing device.

13. The non-transitory computer-readable medium of claim 8, wherein the machine readable instructions, when executed by the processor, further cause the computing device to at least render a user interface on a display connected to the computing device, the user interface providing an option to select the pharmaceutical compound to identify in the blood sample.

14. A computer-implemented method, comprising:
sending a command to a mass spectrometer to analyze a blood sample to generate a mass spectrum;
receiving the mass spectrum from the mass spectrometer;
analyzing the mass spectrum to determine an identity a pharmaceutical compound present in the blood sample; and
generating a report for the blood sample, the report containing the identity of the pharmaceutical compound present in the blood sample.

15. The computer-implemented method of claim 14, wherein analyzing the mass spectrum to determine the identity of the pharmaceutical compound present in the blood sample further comprises:
comparing the mass spectrum to a catalog of mass spectra, the catalog of mass spectra comprising a plurality of entries, each entry representing a mass spectrum of an identified pharmaceutical compound; and
determining that one of the plurality of entries in the catalog of mass spectra matches the mass spectrum received from the mass spectrometer.

16. The computer-implemented method of claim 14, further comprising
receiving a first notification from an auto-sampler of a high-performance liquid-chromatograph that the blood sample has been inserted into the auto-sampler;
in response to receipt of the notification, sending a third command to the high-performance liquid-chromatograph and the mass spectrometer to initiate a self-test; and
determining that the high-performance liquid-chromatograph and the mass spectrometer passed the self-test.

17. The computer-implemented method of claim 16, wherein sending the first command to the mass spectrometer to analyze the blood sample in the auto-sampler to generate the mass spectrum further comprises sending the first command to the mass spectrometer in response to determining that the high-performance liquid-chromatograph and the mass spectrometer passed the self-test.

18. The computer-implemented method of claim 14, further comprising:
rendering a user interface on a display of the computing device; and
sending the report to a remote computing device selected through the user interface rendered on the display of the computing device.

19. The computer-implemented method of claim 14, further comprising rendering a user interface on a display connected to the computing device, the user interface providing an option to select the pharmaceutical compound to identify in the blood sample.
